(19) United States
(12) Patent Application Publication
(10) Pub. No.: US 2008/0311628 A1
(43) Pub. Date: Dec. 18, 2008

(54) Methods and compositions for rapid amplification and capture of nucleic acid sequences

(75) Inventor: Daniel D. Shoemaker, San Diego, CA (US)

Correspondence Address: James P. Broder;Roeder & Broder LLP, 9915 Mira Mesa Blvd. Suite 300, San Diego, CA, 92131, US

(73) Assignee: GHC Technologies, Inc.

(21) Appl. No.: 11/903,014

(22) Filed: Sep. 19, 2007

Related U.S. Application Data
(60) Provisional application No. 60/849,317, filed on Oct. 3, 2006.

(51) Int. Cl. C12P 19/34

(52) U.S. Cl. 435/91.2

(57) ABSTRACT

A method for amplifying a nucleic acid sequence includes the steps of (i) providing a first pair of primers that include one or more uracil nucleotides, the primers being complementary to a portion of a genomic template, (ii) introducing the first pair of primers, the genomic template and a first polymerase into a reaction vessel, (iii) carrying out one or more polymerase chain reaction cycles in the reaction vessel to generate a plurality of first amplicons, and (iv) selectively degrading a portion each first amplicon with a Uracil-DNA Glycosylase to decrease the binding energy of each first amplicon. In one embodiment, the step of selectively degrading includes using a thermostable Uracil-DNA Glycosylase to decrease the binding energy of each first amplicon. In another embodiment, the method also includes the step of adding a second polymerase and a second pair of primers to the reaction vessel to generate a plurality of second amplicons that are different than the first amplicons. Generating the plurality of second amplicons can occur substantially isothermally or non-isothermally. Further, the second pair of primers can be nested primers.

### REFERENCE TO RELATED APPLICATION

This Application claims domestic priority on U.S. Provisional Application Ser. No. 60/849,317, filed on Oct. 3, 2006. The contents of U.S. Provisional Application Ser. Nos. 60/849,317 are incorporated herein by reference to the extent permitted.

### BACKGROUND

Rapid nucleic acid amplification and detection has become increasingly more critical, such as in the areas of biodefense and Point of Care clinical diagnostics. However, efforts to decrease the time required for amplification and analysis of nucleic acid sequences without sacrificing accuracy have not been altogether satisfactory. Although certain processes have been advanced in recent years such as using various isothermal amplification methods, many such methods have drawbacks that are challenging or impossible to overcome. These drawbacks can include difficult and/or slow initiation, limited site selection of primers on a DNA or RNA template and/or suboptimal performance levels. Additionally, conventional polymerase chain reaction (sometimes referred to herein as “PCR”) based amplification methods can require hours to perform and are limited by contamination issues.

### SUMMARY

The present invention is directed toward a method for amplifying a nucleic acid sequence. In one embodiment, the method includes the steps of providing a first pair of primers that include one or more uracil nucleotides, the primers being complementary to a portion of a genomic template; introducing the first pair of primers, the genomic template and a first polymerase into a reaction vessel; carrying out one or more polymerase chain reaction cycles in the reaction vessel to generate a plurality of first amplicons; and selectively degrading a portion each first amplicon with a Uracil-DNA Glycosylase to decrease the binding energy of each first amplicon.

In one embodiment, the step of selectively degrading includes using a thermostable Uracil-DNA Glycosylase to decrease the binding energy of each first amplicon. In another embodiment, the method also includes the step of adding a second polymerase and a second pair of primers to the reaction vessel to generate a plurality of second amplicons that are different than the first amplicons. In this embodiment, the second pair of primers can be different than the first pair of primers. In certain embodiments, generating the plurality of second amplicons occurs substantially isothermally. Alternatively, generating the plurality of second amplicons can occur non-isothermally. In some embodiments, the second amplicons have fewer base pairs than the first amplicons. In another embodiment, each primer in the second pair of primers can include fewer nucleotides than each primer in the first pair of primers. In some embodiments, the second pair of primers are nested primers.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a workflow diagram showing one embodiment of a method including steps for amplification and detection of nucleic acid sequences in accordance with the present invention;

FIG. 2 is a workflow diagram showing an alternative embodiment of a method to initiate the amplification and detection sequence illustrated at Steps 1-3 in FIG. 1;

FIG. 3 is an illustration of one embodiment of a reaction vessel assembly for use during a Microclimate Temperature Exposure (MTE) cycling method to amplify an amplicon; and

FIG. 4 is a graph of experimental results showing generation of Short Amplicon polymerase chain reaction over time, using both temperature cycling and isothermal polymerase chain reaction amplification, as a function of time.

### DESCRIPTION

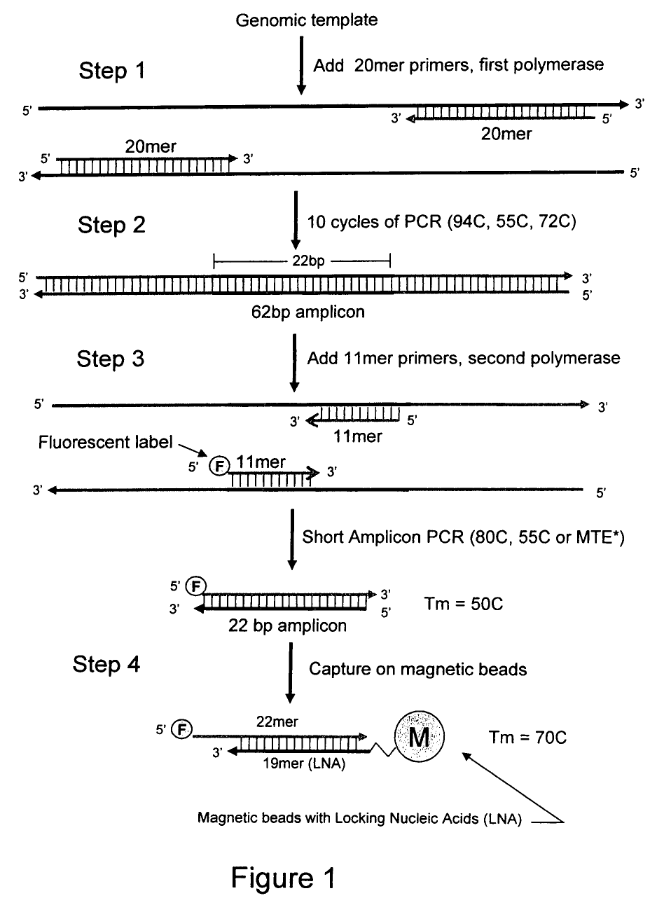

FIG. 1 is a workflow diagram showing one embodiment of a method including steps for amplification and detection of nucleic acid sequences in accordance with the present invention. In the embodiment illustrated in FIG. 1, a genomic template, a first set of two primers (also sometimes referred to herein as a “pair” of primers) and a first polymerase are introduced into a reaction vessel for a plurality of PCR cycles, which can be a reduced number of cycles relative to conventional PCR.

The genomic template can include DNA, RNA or any other suitable nucleic acid sequences. In the embodiment illustrated in FIG. 1, the primer can be a 20-base primer (also referred to herein as a “20mer”). Alternatively, the primer can include greater than or fewer than 20 bases. The first polymerase can include any suitable polymerase known to those skilled in the art of PCR, such as Taq polymerase, as one non-exclusive example.

In the embodiment illustrated in FIG. 1, approximately ten PCR cycles can be carried out, which can result in an approximately 1000-fold amplification of all or a portion of the genomic template, for example. Alternatively, greater or fewer than ten PCR cycles can be used depending upon the desired number of PCR amplicons. In certain embodiments, each PCR cycle includes fluctuating the temperature within a reaction vessel through a plurality of different temperatures to cyclically raise and lower the temperature of the reagents within the reaction vessel. The specific temperatures to be achieved within the reaction vessel depend upon the types of reagents used. For instance, in one non-exclusive example of one PCR cycle, the temperature starts at approximately 94° C., is lowered to 55° C., and then raised to 72° C. In this example, at 94° C., the double-stranded genomic template denatures, generating two single-stranded templates, as illustrated at Step 1 in FIG. 1. At 55° C., the primer anneals (also illustrated at Step 1 in FIG. 1) to the now single-stranded templates, and at 72° C., the polymerase extends the annealed primer (illustrated at Step 2 in FIG. 1). It is recognized that the actual temperatures required for Steps 1 and 2 can vary, and that the temperatures described herein are provided for one specific set of reagents for ease of understanding.

In the embodiment illustrated in FIG. 1, the two primers can be selected based on the length of the amplicon desired, such as an amplicon of approximately 62 base pairs (bp) in length. It is recognized that the amplicon can include any suitable number of bases or base pairs, and that the example illustrated in FIG. 1 is provided as one representative embodiment for ease of understanding and explanation.

At Step 3 in FIG. 1, a second polymerase and a second pair of two “nested” primers are introduced into a reaction mixture. As used herein, a nested primer is positioned inside the primers that are used in the initial round of amplification illustrated at Steps 1 and 2 in FIG. 1. In other words, the 62 bp amplicons that were generated from Steps 1 and 2 serve as templates for this second round of amplification. In one embodiment, the second polymerase is different than the first polymerase. Alternatively, the first polymerase and the second polymerase can be the same.

The specific nucleotides that form the primers in the second set can vary. Further, the length of the primers in the second set can vary. In the embodiment illustrated at Step 3 in FIG. 1, the primers include 11 nucleotides (also referred to as “11mer primers”). Alternatively, the primers can include greater or fewer than 11 nucleotides. Further, in one embodiment, one or more of the primers includes a fluorescent label (illustrated as a circle with an “F”) for easier detection at a later stage of the process.

In this example, from the 62 bp amplicons generated at Step 2 serve as templates for a second round of amplification using nested 11mers as primers which generate a plurality of 22 bp amplicons (also referred to herein as “Short Amplicons”) are produced, as illustrated generally at Step 3 in FIG. 1. In one embodiment, a two-step temperature cycling protocol is used where the temperature fluctuates from 55° C. to 80° C. An alternative temperature cycling method involves using somewhat random temperature fluctuations to drive the amplification. This method is also referred to herein as a “Microclimate Temperature Exposure” (MTE) cycling method, which is described in greater detail below. Alternatively, isothermal amplification or other types of non-isothermal amplification can be utilized to form the Short Amplicons.

At Step 4, magnetic beads containing specially designed capture probes (shown as a 19mer in FIG. 1) are introduced into the reaction mixture. During this step, the 22 bp amplicons become denatured because the reaction temperature is set above the melting temperature (Tₘ) of the amplicon, or by increasing the temperature during PCR to cause denaturing of the double-stranded amplicon. Once the amplicon has denatured, the 22mer strand having the fluorescent label can be captured by one of the capture probes, as shown at Step 4 in FIG. 1.

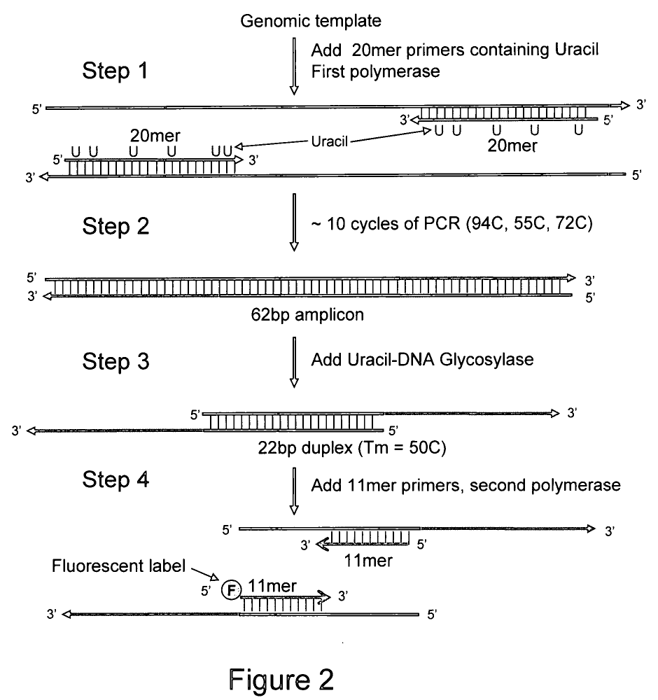

FIG. 2 is a workflow diagram showing an alternative embodiment of a method to initiate the amplification and detection sequence illustrated at Steps 1-3 in FIG. 1. In this embodiment, the double-stranded genomic template, the first set of 20mer primers (or any other suitable length primer) and the first polymerase are added to the reaction vessel at Step 1. In this embodiment, each of the primers includes the use of one or more uracil (U) nucleotides in the place of thymine (T) nucleotides. Although in this embodiment each primer includes five uracil nucleotides, it is recognized that any suitable number of uracil nucleotides can be incorporated into the primers.

At Step 2 in FIG. 2, a plurality of cycles of PCR are carried out as described previously to generate a plurality of 62 bp amplicons.

At Step 3 in FIG. 2, a Uracil-DNA Glycosylase is added to the reaction mixture. The Uracil-DNA Glycosylase functions by removing uracil residues from single-stranded DNA to yield apyrimidic sites. These sites are then susceptible to hydrolysis by heat or alkaline treatment resulting in degradation of the DNA at any uracil-containing sites. Stated another way, the portion of the strand containing the uracil degrades away, leaving the DNA structure illustrated following Step 3 in FIG. 2. It is recognized that other suitably similar methods can be utilized to degrade a particular section of the strand as required. Alternatively, a thermostable Uracil-DNA Glycosylase can be used which allows this reaction to proceed at elevated reaction temperatures (e.g., 60° C.-80° C.).

At Step 4 in FIG. 2, a second polymerase and a second set of nested primers (illustrated as 11mers in FIG. 2) are added to the reaction mixture to ultimately generate the 22 bp amplicon. Removing the uracil-containing portions of the 62 bp amplicon reduces the binding energy of the resulting duplex, which allows the Short Amplicon PCR to proceed more efficiently. This step can be carried out in a somewhat similar manner as Step 3 in FIG. 1.

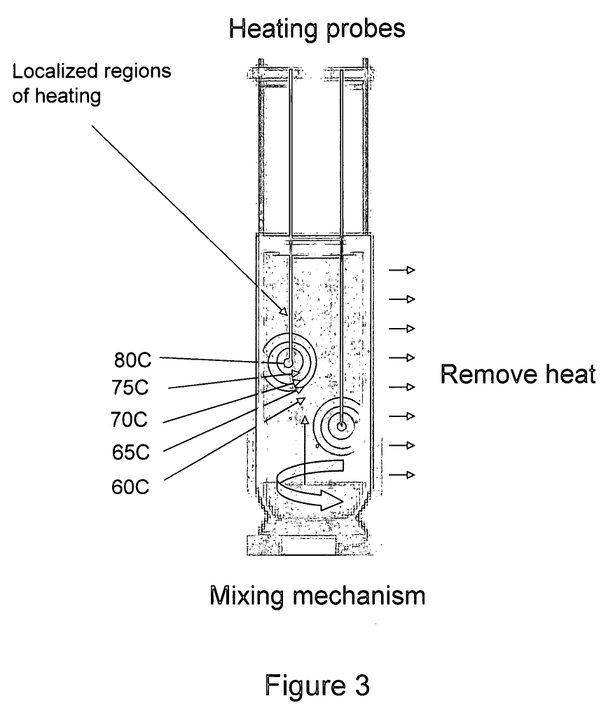

FIG. 3 is an illustration of one embodiment of a reaction vessel assembly for use during the MTE cycling method to amplify the 22 bp amplicon. In the embodiment illustrated in FIG. 3, the reaction vessel assembly includes a reaction vessel and a temperature controller that provides a plurality of temperature microclimates within the reaction vessel. The size of the reaction vessel can vary.

In one embodiment, the temperature controller includes one or more heating probes that introduce localized heat into the reaction mixture. Stated another way, the heating probes are positioned so that they do not uniformly heat the reaction mixture. The heating probes can be positioned at different levels within the reaction vessel as illustrated in FIG. 3, or all at the same level.

In one embodiment, the heating probes can all be heated to a substantially similar or identical temperature. Alternatively, two or more of the heating probes can be heated to different temperatures. As illustrated in FIG. 3, heat can be removed from the reaction vessel at a rate such that the temperatures within the reaction vessel can substantially stabilize without the temperature of the reaction mixture becoming isothermal. In other words, different temperatures are simultaneously and locally maintained within the reaction vessel. By allowing heat to move away from the reaction vessel, the likelihood of a steady increase or decrease in the mean temperature of the reaction mixture within the reaction vessel is reduced or eliminated. In one embodiment, the reaction vessel can be placed at least partially within a cooling vessel (not shown) that can draw heat from the reaction vessel at a desired rate. Alternatively, cooling can be carried out via convection or any other suitable cooling means.

As illustrated in FIG. 3, each of the heating probes generates a continuum of temperatures within the reaction mixture from. For example, one heating probe can produce a temperature of 80° C. Moving away from the heating probe, the temperature gradually lowers to 60° C. Thus, although FIG. 3 only shows five different temperatures for illustrative purposes, the heating probe actually generates a continuum of an infinite number of different temperatures within the reaction vessel.

In one embodiment, the temperature of the heating probes is substantially static. In an alternative embodiment, the temperature of one or more heating probes fluctuates in a rhythmic, cyclical manner. In still another embodiment, the temperature of one or more heating probes fluctuates in a random, non-cyclical manner.

In non-exclusive alternative embodiments, the method of generating different temperature microclimates within the reaction vessel can include the use of lasers or microwave technology for localized heating. Still alternatively, other suitable methods can be utilized for generating and simultaneously maintaining a plurality of different temperatures within the reaction vessel.

In FIG. 3, the reaction vessel assembly can also include a reagent mover (also sometimes referred to herein as a “mixing mechanism”) that moves the reagents through the various temperature microclimates within the reaction vessel. With this design, the reagents (specifically, the amplicons) can be subjected to a random temperature variance depending upon the positioning of the heating probes and the mixing cycle of the reagents by the reagent mover. In one embodiment, the reagent mover can be a mechanism that rotates or otherwise moves the reaction vessel without substantial movement of the temperature controller. Alternatively, the reaction vessel can remain substantially stationery while the temperature controller is moved, i.e. rotated, oscillated or moved in an up and down or side-to-side motion, as non-exclusive examples.

It is recognized that other methods for moving the reagents within the reaction vessel can be utilized. In non-exclusive alternative embodiments, the reaction vessel can be vibrated, oscillated or otherwise moved to move the reagents within the reaction vessel. Still alternatively, a mixing device (separate from the temperature controller) can be introduced into the reaction vessel and moved in a manner to stir or otherwise move the reagents, such as a magnetic stir bar or any other suitable device. Any other suitable means of mixing or stirring the reagents can be used.

With the designs provided herein, the reagents are somewhat randomly moved through a continuum of different temperatures. Each reagent can have its own optimal temperature at which certain processes occur, such as denaturing, annealing, binding and extending. Because of the continuum of temperatures provided within the reaction vessel, the likelihood is increased that each reagent will encounter its optimal temperature for a given stage of the reaction process. Consequently, the reagents within the reaction vessel can simultaneously be at different stages of the amplification process because of the different temperatures within the reaction vessel. For example, while one double-stranded amplicon may be in the process of denaturing, a primer may be annealing on an already denatured single strand, while extension of a primer may be simultaneously occurring on yet another single strand. Stated another way, with this design, multiple stages of the amplification process occur concurrently.

The reaction vessel assembly provided herein generates localized temperature microclimates within the reaction vessel for better performing the necessary functions of this stage of amplification. In certain embodiments, the temperature controller provides a somewhat more narrow range of temperatures than is used during typical non-isothermal PCR. For example, in one embodiment, the temperature of the reagents generated by the temperature controller can range from approximately 50° C. to approximately 80° C. In non-exclusive alternative embodiments, the temperature range can be between approximately 55° C. and approximately 80° C., approximately 60° C. and approximately 80° C., approximately 50° C. and approximately 75° C., approximately 50° C. and approximately 72° C., approximately 50° C. and approximately 85° C., or approximately 50° C. and approximately 90° C. Still alternatively, the temperature range can be outside of or narrower than the above-referenced ranges, as determined by the requirements of the specific type of amplification being performed.

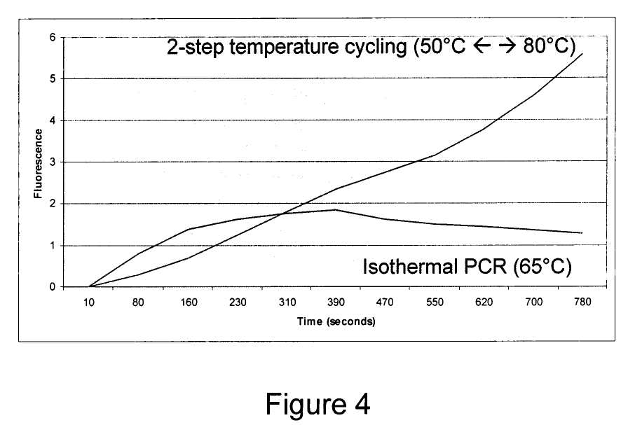

FIG. 4 is a graph of experimental results showing generation of Short Amplicon PCR over time, using (i) temperature cycling between 50° C. and 80° C., and (ii) isothermal PCR amplification at 65° C., as a function of time. Fluorescence is a means of measuring the quantity of amplification product generated. The graph indicates that at approximately 350 seconds, the isothermal PCR method results in a substantial plateau of amplification product, while the amplification product using the temperature cycling method continues to increase.

Referring back to FIG. 1, at Step 4, once the 22mer has denatured, the single strands having the fluorescent labels are captured the amplified 22mer products (or products of any suitable number of nucleotides). In this embodiment, one or more capture probes (illustrated as “19mer (LNA)” in FIG. 1) are used to capture the desired strand of the amplified 22mer product. The capture probes include a series of bases that are complementary to at least a portion of the 22mer product.

In one embodiment, the capture probes can extend directly or indirectly from magnetic beads (indicated as an “M” in a circle), as one non-exclusive example. In this example, at greater than 50° C. (other suitable temperatures can be used), the double stranded 22mer becomes denatured, and the desired strand can bind to the capture probe. In certain embodiments, the capture probes can include one or more locked nucleic acids (LNA's). One example of a more detailed explanation of LNA's can be found in publications known to those skilled in the art, including, but not limited to “Locked Nucleic Acids (LNA) (Ørum, H., Jakobsen, M. H., Koch, T., Vuust, J. and Borre, M. B. (1999) Detection of the Factor V Leiden Mutation by Direct Allele-specific Hybridization of PCR Amplicons to Photoimmobilized Locked Nucleic Acids. Clin Chem., 45:1898-1905)”, the publication of which is incorporated herein by reference to the extent permitted.

While the particular methods and compositions for rapid amplification and/or capturing of nucleic acid sequences as shown and disclosed herein are fully capable of obtaining the objects and providing the advantages herein before stated, it is to be understood that it is merely illustrative of the presently preferred embodiments of the invention and that no limitations are intended to the details of the methods, construction or design herein shown and described.

What is claimed is:

1. A method for amplifying a nucleic acid sequence, the method comprising the steps of:
providing a first pair of primers that include one or more uracil nucleotides, the primers being complementary to a portion of a genomic template;
introducing the first pair of primers, the genomic template and a first polymerase into a reaction vessel;
carrying out one or more polymerase chain reaction cycles in the reaction vessel to generate a plurality of first amplicons; and
selectively degrading a portion each first amplicon with a Uracil-DNA Glycosylase to decrease the binding energy of each first amplicon.

2. The method of claim 1 wherein the step of selectively degrading includes using a thermostable Uracil-DNA Glycosylase to decrease the binding energy of each first amplicon.

3. The method of claim 1 further comprising the step of adding a second polymerase and a second pair of primers to the reaction vessel to generate a plurality of second amplicons that are different than the first amplicons, the second pair of primers being different than the first pair of primers.

4. The method of claim 3 wherein generating the plurality of second amplicons occurs substantially isothermally.

5. The method of claim 3 wherein generating the plurality of second amplicons occurs non-isothermally.

6. The method of claim 3 wherein the second amplicons have fewer base pairs than the first amplicons.

7. The method of claim 3 wherein each primer in the second pair of primers includes fewer nucleotides than each primer in the first pair of primers.

8. The method of claim 3 wherein the second pair of primers are nested.

9. A method for amplifying a nucleic acid sequence, the method comprising the steps of:
providing a first pair of primers that include one or more uracil nucleotides, the primers being complementary to a portion of a genomic template, the primers each having at least n nucleotides;
introducing the first pair of primers, the genomic template and a first polymerase into a reaction vessel;
carrying out one or more polymerase chain reaction cycles in the reaction vessel to generate a plurality of first amplicons;
selectively degrading a portion each first amplicon with a Uracil-DNA Glycosylase to decrease the binding energy of each first amplicon; and
adding a second polymerase and a second pair of primers to the reaction vessel to generate a plurality of second amplicons that are different than the first amplicons, the second pair of primers each having fewer than n nucleotides.

10. The method of claim 9 wherein the step of selectively degrading includes using a thermostable Uracil-DNA Glycosylase to decrease the binding energy of each first amplicon.

11. The method of claim 9 wherein generating the plurality of second amplicons occurs substantially isothermally.

12. The method of claim 9 wherein generating the plurality of second amplicons occurs non-isothermally.

13. The method of claim 9 wherein the second amplicons have fewer base pairs than the first amplicons.

14. The method of claim 9 wherein the second pair of primers are nested.

15. A method for amplifying a nucleic acid sequence, the method comprising the steps of:
providing a first pair of primers that include one or more uracil nucleotides, the primers being complementary to a portion of a genomic template;
introducing the first pair of primers, the genomic template and a first polymerase into a reaction vessel;
carrying out one or more polymerase chain reaction cycles in the reaction vessel to generate a plurality of first amplicons having at least n base pairs;
selectively degrading a portion each first amplicon with a Uracil-DNA Glycosylase to decrease the binding energy of each first amplicon; and
adding a second polymerase and a second pair of primers to the reaction vessel to generate a plurality of second amplicons, each second amplicon having fewer than n base pairs.

16. The method of claim 15 wherein the step of selectively degrading includes using a thermostable Uracil-DNA Glycosylase to decrease the binding energy of each first amplicon.

17. The method of claim 15 wherein generating the plurality of second amplicons occurs substantially isothermally.

18. The method of claim 15 wherein generating the plurality of second amplicons occurs non-isothermally.

19. The method of claim 15 wherein each primer in the second pair of primers includes fewer nucleotides than each primer in the first pair of primers.

20. The method of claim 15 wherein the second pair of primers are nested.
